(19) United States
(12) Patent Application Publication
(10) Pub. No.: US 2020/0121266 A1
(43) Pub. Date: Apr. 23, 2020

(54) CARRYING OUT AN IMAGING SCAN OF A PATIENT IN A COMPUTED TOMOGRAPHY SYSTEM

(71) Applicant: Siemens Healthcare GmbH, Erlangen (DE)

(72) Inventor: Christian HOFMANN, Erlangen (DE)

(73) Assignee: Siemens Healthcare GmbH, Erlangen (DE)

(21) Appl. No.: 16/597,269

(22) Filed: Oct. 9, 2019

(30) Foreign Application Priority Data
Oct. 18, 2018 (DE) 10 2018 217 888.7

(51) Int. Cl. A61B 6/03; A61B 6/00; A61B 5/00; A61B 5/08; G06T 11/00

(52) U.S. Cl. CPC A61B 6/032; A61B 6/5217; G06T 2210/41; A61B 5/08; G06T 11/003; A61B 5/0033

(57) ABSTRACT

A method is for carrying out an imaging scan of a patient in a computed tomography system. In an embodiment, the method includes acquiring a respiratory cycle of the patient; providing the respiratory cycle as a reference respiratory cycle; selecting at least one respiratory phase of the reference respiratory cycle, shorter than a cycle period of the reference respiratory cycle; and carrying out the imaging scan in the computed tomography system. A respiration of the patient is acquired and is transferred as a respiratory signal progression; the respiratory signal progression is compared with the at least one selected respiratory phase of the reference respiratory cycle, by which a binary comparison result is generated; and dependent upon the binary comparison result, a data acquisition is triggered in the computed tomography system, by which projection data is acquired in the at least one respiratory phase.

### PRIORITY STATEMENT

The present application hereby claims priority under 35 U.S.C. § 119 to German patent application number DE 102018217888.7 filed Oct. 18, 2018, the entire contents of which are hereby incorporated herein by reference.

### FIELD

Embodiments of the invention generally relate to a method for carrying out an imaging scan of a patient in a computed tomography system, the computed tomography system and an associated computer program product.

### BACKGROUND

Radiotherapy planning for a patient typically requires a provision of a medical image in a specific respiratory phase of the patient, for example, if the radiotherapy planning relates to a target volume that is moving due to the respiration of the patient, in particular, a lung and/or abdominal carcinoma. In this case, an image of the target volume of the radiotherapy planning in the medical image depends on the respiration, in particular, on the respiratory phase. The radiotherapy planning is preferably adapted to the respiratory phase of the medical image.

In a conventional imaging scan, typically projection data is acquired for a whole respiratory cycle of the respiration, specifically for each respiratory phase of the respiratory cycle, and only those respiratory phases for which the medical image is to be provided are filtered out during a reconstruction of the projection data.

### SUMMARY

At least one embodiment of the invention provides a method for carrying out an imaging scan of a patient in a computed tomography system, an associated computed tomography system and an associated computer program product, with which a dosage burden on the patient is reduced.

Advantageous embodiments are disclosed in the subclaims.

At least one embodiment of the invention is directed to a method for carrying out an imaging scan of a patient in a computed tomography system, comprising:

acquiring a respiratory cycle of the patient via a respiratory detection unit of the computed tomography system,

providing the respiratory cycle of the patient in a planning unit of the computed tomography system as a reference cycle,

selecting at least one respiratory phase of the reference respiratory cycle via the planning unit, whereby the at least one selected respiratory phase is shorter than a cycle period of the reference respiratory cycle,

transferring the at least one selected respiratory phase together with the reference respiratory cycle to a control unit of the computed tomography system,

carrying out the imaging scan in the computed tomography system,

wherein a respiration of the patient is acquired via the respiratory detection unit and is transferred as a respiratory signal progression into the control unit,

wherein the respiratory signal progression is compared in the control unit with the at least one selected respiratory phase of the reference respiratory cycle, by which a binary comparison result is generated,

wherein, dependent upon the binary comparison result, a data acquisition is triggered in the computed tomography system, whereby projection data is acquired in the at least one respiratory phase.

At least one embodiment of the invention is directed to a method for carrying out an imaging scan of a patient in a computed tomography system, comprising:

acquiring a respiratory cycle of the patient via a respiratory detection unit of the computed tomography system,

providing the respiratory cycle of the patient, in a planning unit of the computed tomography system, as a reference respiratory cycle;

selecting, via the planning unit, at least one respiratory phase of the reference respiratory cycle, the at least one respiratory phase selected being relatively shorter than a cycle period of the reference respiratory cycle;

transferring the at least one respiratory phase selected, together with the reference respiratory cycle, into a control unit of the computed tomography system; and

carrying out the imaging scan in the computed tomography system,
a respiration of the patient being acquired via the respiratory detection unit and being transferred as a respiratory signal progression into the control unit,
the respiratory signal progression being compared in the control unit with the at least one respiratory phase selected, of the reference respiratory cycle, by which a binary comparison result is generated, and
wherein, dependent upon a result of the binary comparison, a data acquisition is triggered in the computed tomography system by which projection data is acquired in the at least one respiratory phase.

At least one embodiment of the application is directed to a computed tomography system comprising the respiratory detection unit, the planning unit and the control unit. Typically, the computed tomography system comprises the X-ray tube and the X-ray detector. The computed tomography system is preferably configured according to at least one embodiment of the method for carrying out the imaging scan of the patient.

At least one embodiment of the application is directed to a computed tomography system, comprising:
a respiratory detection unit to acquire a respiratory cycle of a patient;
a planning unit to provide a respiratory cycle of the patient, as a reference respiratory cycle and to
select at least one respiratory phase of the reference respiratory cycle, the at least one respiratory phase selected being relatively shorter than a cycle period of the reference respiratory cycle; and
a control unit to receive the at least one respiratory phase selected transferred from the planning unit, together with the reference respiratory cycle and to control carrying out an imaging scan in the computed tomography system, wherein
   a respiration of the patient is acquired via the respiratory detection unit and is transferred as a respiratory signal progression into the control unit,
   the respiratory signal progression is compared in the control unit with the at least one respiratory phase selected, of the reference respiratory cycle, by which a binary comparison result is generated, and
   dependent upon a result of the binary comparison, a data acquisition is triggered in the computed tomography system by which projection data is acquired in the at least one respiratory phase.

At least one embodiment of the application is directed to a computer program product which is directly loadable into a memory store of the computer unit has program code segments in order to implement at least one embodiment of the method for carrying out the imaging scan of the patient in the computed tomography system when the computer program product is executed in the computer unit. The computer unit can be part of the computed tomography system and/or can comprise the processor and the working memory.

At least one embodiment of the application is directed to a non-transitory computer program product, storing a computer program which is directly loadable into a memory store of a computer unit, the computer program including program code segments to carry out at least one embodiment of the method when the computer program is executed in the computer unit.

At least one embodiment of the application is directed to a non-transitory computer readable medium, storing a computer program which is directly loadable into a memory store of a computer unit, the computer program including program code segments to carry out at least one embodiment of the method when the computer program is executed in the computer unit.

The computer program product can be a computer program or can comprise a computer program. The computer program product has, in particular, the program code segments which form at least one embodiment of the inventive method. By this, at least one embodiment of the inventive method can be carried out in a defined and repeatable manner and monitoring of a passing on of at least one embodiment of the inventive method can be achieved.

The computer program product is preferably configured such that the computer unit can carry out at least one embodiment of the inventive method via the computer program product. The program code segments can be loaded, in particular, into a memory store of the computer unit and are typically carried out via a processor of the computer unit with access to the memory store. If the computer program product, in particular the program code segments, is carried out in the computer unit, typically all the inventive embodiments of the method described can be carried out.

The computer program product is, for example, a physical computer-readable medium and/or includes a program stored digitally as a data packet in a computer network. The computer program product can represent the physical, computer-readable medium and/or the data packet in the computer network. At least one embodiment of the invention can thus also proceed from the physical computer-readable medium and/or the data packet in the computer network.

### BRIEF DESCRIPTION OF THE DRAWINGS

The invention will now be described and explained in greater detail making reference to the example embodiments illustrated in the drawings. In principle, structures and units which remain essentially the same are identified in the following description of the figures with the same reference signs as in the first occurrence of the relevant structure or unit.

In the drawings:

FIG. 1 is a method for carrying out an imaging scan of a patient in a computed tomography system in a first example embodiment,

FIG. 2 is the method in a second example embodiment,

FIG. 3 is a schematic representation of the reference respiratory cycle in a third example embodiment,

FIG. 4 is a schematic representation of the reference respiratory cycle and of the respiratory signal progression in a fourth example embodiment, and

FIG. 5 is a computed tomography system.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the invention is directed to a method for carrying out an imaging scan of a patient in a computed tomography system, comprising:

acquiring a respiratory cycle of the patient via a respiratory detection unit of the computed tomography system,

providing the respiratory cycle of the patient in a planning unit of the computed tomography system as a reference cycle,

selecting at least one respiratory phase of the reference respiratory cycle via the planning unit, whereby the at least one selected respiratory phase is shorter than a cycle period of the reference respiratory cycle,

transferring the at least one selected respiratory phase together with the reference respiratory cycle to a control unit of the computed tomography system,

carrying out the imaging scan in the computed tomography system,

wherein a respiration of the patient is acquired via the respiratory detection unit and is transferred as a respiratory signal progression into the control unit,

wherein the respiratory signal progression is compared in the control unit with the at least one selected respiratory phase of the reference respiratory cycle, by which a binary comparison result is generated,

wherein, dependent upon the binary comparison result, a data acquisition is triggered in the computed tomography system, whereby projection data is acquired in the at least one respiratory phase.

The method offers, in particular, the following advantages:

Preferably, a scan duration of the imaging scan is shortened in that the execution of the imaging scan can be ended after the projection data is acquired in the at least one respiratory phase. The shortening of the scan duration of the imaging scan is advantageous, in particular, since typically the scan duration correlates to the costs for the imaging scan. The shortened scan duration can, in particular, increase a level of comfort for the patient.

A further advantage can be that the binary comparison result preferably enables a dosage modulation of the imaging scan dependent upon the respiration of the patient. The dosage modulation can enable a selective acquisition of the projection data in the at least one respiratory phase, which can be advantageous as compared with a conventional continuous acquisition of projection data. The selective acquisition can be designated “flexible pulsing”. The dosage modulation further preferably enables projection data to be acquired for particular respiratory phases, in particular non-acquirable respiratory phases of the reference respiratory cycle, with a comparatively lower dose burden or no projection data. The binary comparison result enables, in particular, a “gating” of the at least one respiratory phase.

Advantageously, before the imaging scan is carried out, the at least one respiratory phase can be selected such that conventional projection data is not acquired for the further, in particular not selected, respiratory phases, but preferably only the projection data in the at least one respiratory phase. The selection of the at least one respiratory phase can preferably take place dependent upon a radiotherapy planning.

The comparison of the respiratory signal progression with the at least one selected respiratory phase of the reference respiratory cycle thereby enables, in particular, an increase in a data quality of the projection data since preferably a respiration divergence, for example, a respiratory artifact and/or an irregular respiration, can be determined whilst advantageously no projection data is acquired.

The abbreviation of the scan duration, the selection of the at least one respiratory phase and/or the binary comparison result enable, in particular, an advantageous adaptation of the dosage burden to the patient, particularly advantageously a reduction in the dosage burden on the patient.

The imaging scan can be carried out, in particular, dependent upon a clinical objective. The clinical objective can relate to the radiotherapy planning and/or the respiration of the patient. For example, the projection data in the at least one respiratory phase can be used for a provision of a ventilation map and/or a respiration-correlated volumetric image data set of the patient.

The respiratory detection unit can comprise a respiratory belt, a spirometer and/or a camera. The camera can be, in particular, a 3D camera and/or a video camera and/or an infrared camera. It is, in principle, conceivable that for the acquisition of the respiratory cycle and for the acquisition of the respiration, different units of the respiratory detection unit are used. The patient can be positioned for the acquisition of the respiratory cycle on a patient support of the computed tomography system and remain there for the execution of the imaging scan. In other words, the patient is preferably not repositioned between the acquisition of the respiratory cycle and the execution of the imaging scan, but remains, in particular, on the patient support.

Typically, a respiration of the patient is acquired via the respiratory detection unit at least over a cycle period of the respiration of the patient. The cycle period is typically 2n if the respiration is parameterized in a phase angle-dependent manner or cyclically. The respiratory cycle comprises, in particular, those respiratory phases which together form the cycle period. In principle, it is conceivable that the respiratory cycle is acquired until a particular respiratory phase of the respiratory cycle is acquired again. The particular respiratory phase of the respiratory cycle can characterize an inhalation and/or an exhalation. Preferably, the respiration cycle of the patient is acquired until the reference respiratory cycle preferably has the cycle period of the respiration. The respiratory cycle is preferably acquired, in particular, during a regular respiration of the patient, typically without the respiratory artifact and/or the respiration divergence.

The provision of the respiratory cycle of the patient can comprise a transfer of the acquired respiratory cycle into the planning unit. The reference respiratory cycle is typically displayed on a display unit of the planning unit.

The provision of the respiratory cycle comprises, in particular, the display. The reference respiratory cycle comprises, for example, the cycle period. Typically, a cycle period of the reference respiratory cycle corresponds to the cycle period of the respiration of the patient. For example, particular repeating respiratory phases of the respiratory cycle are provided in the planning unit only once. The reference respiratory cycle typically covers 2π if the reference respiratory cycle is parameterized in a phase angle-dependent manner or cyclically. In particular, the reference respiratory cycle comprises the cycle period. In principle, it is conceivable that the reference respiratory cycle is provided to be shorter or longer than the cycle period. The provision can comprise a shortening and/or an interpolation and/or an extrapolation and/or a modeling and/or a filtration of the acquired respiratory cycle. The reference respiratory cycle is preferably respiration artifact-free.

The at least one selected respiratory phase is shorter than a cycle period of the reference respiratory cycle. In particular, the at least one selected respiratory phase covers a smaller phase angle than the cycle period of the reference respiratory cycle, in particular, less than 2π. This is advantageous to the extent that the projection data can be acquired before the cycle period of the reference respiratory cycle ends, so that the execution of the imaging scan can preferably be ended sooner.

The selection of the at least one respiratory phase preferably takes place dependent upon the clinical objective, for example, the radiotherapy planning. The selection takes place, for example, automatically or by a user of the planning unit, for which purpose, the planning unit can, for example, have input device(s). The selection of the at least one respiratory phase can take place, for example, such that the user marks, via the input device(s), the at least one respiratory phase relative to the reference respiratory cycle on the display unit, in particular using drag-and-drop. It is conceivable that the reference respiratory cycle is segmented into different respiratory phases before the user marks the at least one respiratory phase.

Alternatively or additionally, the user can select, for example, the at least one respiratory phase from a list with different respiratory phases according to the segmentation of the reference respiratory cycle. The at least one selected respiratory phase can be selected, for example, relative to the reference respiratory cycle. The at least one selected respiratory phase typically covers the reference respiratory cycle, in particular the cycle period of the reference respiratory cycle, at least partially.

The at least one selected respiratory phase is preferably transferred, together with the reference respiratory cycle, from the planning unit into the control unit. The transfer can involve a calling of the at least one selected respiratory phase, together with the reference respiratory cycle, from the planning unit. The computed tomography system can comprise a storage unit in which, for example, the reference respiratory cycle, the acquired respiratory cycle and/or the at least one selected respiratory phase can be temporarily stored. The storage unit is linked, in particular, to the respiratory detection unit, the planning unit and/or the control unit of the computed tomography system, for example, via a network.

The control unit is configured, in particular, for the execution of a scan protocol which represents the execution of the imaging scan of the patient in the computed tomography system, dependent upon the at least one selected respiratory phase and upon the reference cycle. It is conceivable, in principle, that via the planning unit, the scan protocol which, in particular, parameterizes the imaging scan, is defined.

The scan protocol can have, for example, a scan region with one or more z-positions, a contrast medium administration, a matrix size, a table advance, a tube current, a tube voltage, a reconstruction kernel and/or an X-ray collimation, in particular, as parameterization. The at least one respiratory phase selected and/or the reference respiratory cycle can be part of the scan protocol. The scan protocol is specified, for example, by the user or automatically, dependent upon the clinical objective. Dependent upon the parameterization of the scan protocol, the scan duration of the imaging scan typically varies. The scan protocol can be represented, in particular, in scan protocol program code segments which can be implemented, preferably in the control unit.

The control unit can comprise control information which can be at least partially carried out, for example, in a processor and/or in a working memory of a computer unit. The control information can comprise the scan protocol program code segments.

In general, a respiratory signal typically comprises an amplitude and a time point. The respiratory detection unit is typically used for the acquisition of the respiratory cycle of the patient and for the acquisition of the respiration of the patient. The acquisition of the respiration of the patient comprises, in particular, a regular and/or a continuous acquisition of respiratory signals of the respiration. The acquisition of the respiration can depend upon a sampling rate. The sampling rate is, in particular, between 1 and 100 Hz, particularly advantageously between 40 and 60 Hz.

Typically, a plurality of respiratory signals is acquired per cycle period of the respiration. The respiratory signal progression typically comprises the plurality of respiratory signals, for example in a vector or a list. The respiratory signal progression comprises, in particular, respiratory signals of a plurality of respiration cycles of the respiration. The respiratory signal progression is typically updated continually during the execution of the imaging scan. The respiration of the patient is preferably acquired online or live during the execution of the imaging scan. The respiratory signal progression is transferred, for example, from the respiratory detection unit via the network into the control unit.

The comparison of the respiratory signal progression with the at least one selected respiratory phase of the reference respiratory cycle can comprise a shortening and/or an interpolation and/or an extrapolation and/or a modeling and/or a filtration of the respiratory signal progression. The comparison comprises, in particular, a stipulation of criteria on the basis of the at least one selected respiratory phase. The generation of the binary comparison result comprises, in particular, a checking of whether the respiratory signal progression relative to the at least one selected respiratory phase fulfills the criteria or not.

The criteria comprise, in particular, a tolerance range for a divergence between the respiratory signal progression and the at least one selected respiratory phase. The divergence can comprise, in particular, a temporal divergence between the respiratory signal progression, in particular, a most recent respiratory signal of the respiratory signal progression and the at least one selected respiratory phase.

For example, a time point of the most recent respiratory signal of the respiratory signal progression relative to the respiratory cycle of the patient can be determined and compared with the at least one selected respiratory phase, whereby the temporal divergence is determined and compared with the tolerance range, with the result that the binary comparison result is generated. Alternatively or additionally, the divergence can comprise a divergence in a regularity of the respiratory signal progression and in a regularity of the at least one selected respiratory phase. The regularity can be determined, in particular, by way of the modeling and/or the filtration and/or the interpolation and/or quantified.

The comparison can comprise a comparison of the quantified regularity. For example, the tolerance range can be specified such that, on the basis of the irregular respiration of the patient, no data acquisition takes place and/or only during the regular respiration of the patient does the data acquisition take place. Due to the irregular respiration and/or the respiration artifact and/or the respiration divergence, the regularity can be lower than in comparison with the regularity during the regular respiration. In a particularly preferred embodiment, the divergence can comprise the temporal divergence and the divergence in the regularity.

The binary comparison result typically comprises a True for the data acquisition if the respiratory signal progression relative to the at least one selected respiratory phase fulfills the criteria. The binary comparison result can have different values, in particular True or False and/or, in particular, during the execution of the imaging scan, can change a plurality of times. The changing of the binary comparison result during the imaging scan can depend upon the sampling rate during the acquisition of the respiration of the patient and/or can be clocked by a clock unit of the control unit. It is conceivable, in principle, that dependent upon the sampling rate or the clock unit, the binary comparison result has True or False a plurality of times before it changes. The binary comparison result can have a True or a False, for example, for each comparison of the respiratory signals of the respiratory signal progression, in particular, in a vector or a list.

The data acquisition takes place, for example, via an X-ray tube and an X-ray detector. The X-ray tube is preferably switched on dependent upon the binary comparison result, whereby typically, X-ray radiation is emitted, or is switched off. It is conceivable, in principle, that the X-ray radiation is switched on or off via a collimator in the beam path of the computed tomography system. The data acquisition typically takes place by way of the X-ray radiation.

The triggering of the data acquisition typically corresponds to the switching on of the X-ray radiation, in particular if the binary comparison result has True. The data acquisition is typically ended or not triggered, whereby, for example, the X-ray radiation is switched off if the binary comparison result has False.

If the binary comparison result has False for the data acquisition, typically the X-ray radiation is switched off, for example, via the collimator or the X-ray tube. In this case, the X-ray radiation is typically absorbed completely outside the patient. In other words, the patient is typically not irradiated with the X-ray radiation if the binary comparison result has a False, so that, for example, the dosage burden from the X-ray radiation is reduced. The dosage burden on the patient is preferably only increased during the at least one selected respiratory phase of the patient.

The switching on and/or the switching off of the X-ray radiation preferably takes place within 1 s, particularly preferably within 0.1 s or in a preferred case, instantaneously after the triggering of the data acquisition. The control unit typically triggers the data acquisition dependent upon the binary comparison result. Advantageously, the control unit can generate the binary comparison result after the sampling of the most recent respiratory signal and trigger the data acquisition or switch off the X-ray radiation before, according to the sampling rate, a further respiratory signal of the respiration is acquired and/or is transferred into the control unit for the comparison. The triggering of the data acquisition and/or the switching off of the X-ray radiation can take place multiple times if the binary comparison result changes multiple times. The switching on and/or switching off of the X-ray radiation typically takes place according to the sampling rate on acquisition of the respiration of the patient and/or according to the clock rate of the control unit.

Typically, the data acquisition is triggered for a data acquisition period which is specified, for example, dependent upon the clinical objective and/or is prolonged or ended by way of the further respiratory signal in accordance with the respiratory signal progression.

During the data acquisition, typically the projection data is acquired, for which, typically, the X-ray radiation at least partially penetrates the patient and/or is at least partially detected by the X-ray detector. The projection data is typically raw data of the computed tomography system. The projection data can be temporarily stored, for example, in the storage unit of the computed tomography system and/or transferred into a radiology information system and/or into a PACS image archiving system.

One embodiment provides that after the imaging scan has been carried out, a medical image is reconstructed and provided in the at least one respiratory phase, making use of the projection data. The reconstruction of the projection data can take place, for example, in the control unit. The reconstruction can be represented in reconstruction program code segments. For the reconstruction, the control unit can, for example, call up the projection data from the X-ray detector and/or from the memory unit and/or the radiology information system and/or the PACS image archiving system. The reconstruction takes place, for example, using the reconstruction kernel and/or a filtered back projection and/or an iterative reconstruction rule. The medical image is provided, for example, in the radiology information system and/or in the PACS image archiving system and/or on the planning unit. The provision on the planning unit can comprise a display of the medical image for the user of the planning unit. The medical image preferably has a higher image quality and can advantageously be provided faster than in a conventional medical imaging. The medical image is present, for example, according to a DICOM image format.

One embodiment provides that the at least one respiratory phase comprises a first respiratory phase and a second respiratory phase which cover different respiratory phases of the reference respiratory cycle, and wherein the data acquisition is triggered according to the first respiratory phase and according to the second respiratory phase. In this case, typically a total of the first respiratory phase and of the second respiratory phase is shorter than the cycle period of the reference respiratory cycle. The different respiratory phases can comprise the inhalation, the exhalation and/or an arbitrary number of intermediate phases between the inhalation and the exhalation.

The transfer of the at least one selected respiratory phase and/or the execution of the imaging scan and/or the comparison of the at least one selected respiratory phase typically relate to the first respiratory phase and the second respiratory phase.

The binary comparison result can change, for example, between the first respiratory phase and the second respiratory phase to False, wherein during the first respiratory phase and/or the second respiratory phase, the binary comparison result has True. If the first respiratory phase and the second respiratory phase cover the different respiratory phases of the reference respiratory cycle, an overlap between the first respiratory phase and the second respiratory phase is typically empty.

One embodiment provides that the imaging scan is carried out in a scan region with a first z-position and a second z-position and whereby the projection data is acquired according to the at least one respiratory phase at the first z-position and at the second z-position. The transfer of the at least one selected respiratory phase and/or the execution of the imaging scan and/or the comparison of the at least one selected respiratory phase typically relate to the first z-position and the second z-position. The first z-position and the second z-position are specified, for example, by way of the scan protocol. This embodiment is advantageous, in particular, because the projection data can be acquired at different z-positions in the at least one respiratory phase, whereby a medical image can be reconstructed and/or provided at the first z-position and at the second z-position which can comprise a volumetric medical image data set.

One embodiment provides that the at least one respiratory phase comprises the first respiratory phase and the second respiratory phase, which cover the different respiratory phases of the reference respiratory cycle, whereby the imaging scan is carried out in the scan region with the first z-position and the second z-position, whereby the data acquisition is triggered according to the first respiratory phase and according to the second respiratory phase and whereby the projection data is acquired according to the at least one respiratory phase at the first z-position and at the second z-position. This embodiment enables, in particular, the acquisition of the projection data such that the respiration-correlated volumetric image data set can be reconstructed and provided. The respiration-correlated image data set is based, in particular, on projection data which is acquired at different z-positions in a plurality of respiratory phases of the reference respiratory cycle.

One embodiment provides that, for the comparison of the reference respiratory cycle, parameterization takes place cyclically dependent upon a rate of change of the reference respiratory cycle and an amplitude of the reference respiratory cycle and whereby the respiratory signal progression is parameterized cyclically dependent upon a rate of change of the respiratory signal progression and an amplitude of the respiratory signal progression. The rate of change describes, in particular, a speed and/or can be calculated, for example, via an amplitude change per time unit. The amplitude of the reference respiratory cycle and/or the amplitude of the respiratory signal progression can be, for example, normalized and/or weighted. The cyclical parameterization is, in particular, advantageous since the reference respiratory cycle preferably has the cycle period and/or since the respiration of the patient is typically periodic. The cyclical parameterization can correspond to the phase angle-dependent parameterization.

One embodiment provides that the comparison of the respiratory signal progression with the at least one selected respiratory phase of the reference respiratory cycle comprises a calculation of a tangent of the respiratory signal progression. The calculation of the tangent is, in particular, enabled in that the respiratory signal progression has more than one respiratory signal. For the calculation of the tangent, typically a plurality of respiratory signals of the respiratory signal progression is taken into account which are, for example, averaged or weighted. Typically, the respiratory signal progression has just at the beginning of the imaging scan only one respiratory signal.

One embodiment provides that the comparison of the respiratory signal progression with the at least one selected respiratory phase of the reference respiratory cycle comprises a calculation of a vector between the respiratory signal progression and a central point of the reference respiratory cycle. The central point of the reference respiratory cycle can depend on an integral under the reference respiratory cycle. In particular, if the reference respiratory cycle is cyclically parameterized, the central value can be formed from y and x values of the reference respiratory cycle. It is, in principle, conceivable that the y and x values of the reference respiratory cycle are differently weighted, whereby the central point of the reference respiratory cycle is calculated.

One embodiment provides that the comparison of the respiratory signal progression with the at least one selected respiratory phase of the reference respiratory cycle comprises a specification of an angular range according to the at least one selected respiratory phase of the reference respiratory cycle and whereby the angular range is specified dependent upon the rate of change of the reference respiratory cycle and the amplitude of the reference respiratory cycle. The specification of the angular range comprises, in particular, a conversion of the at least one selected respiratory phase into the angular range, in particular, if the reference respiratory cycle is cyclically parameterized.

One embodiment provides that a tangential angular range is specified dependent upon the angular range about a reference tangent of the reference respiratory cycle, whereby on comparison, the tangent of the respiratory signal progression and the tangential angular range are used. This embodiment is, in particular, advantageous since the tangent and the tangential angular range can typically be rapidly compared.

One embodiment provides that a vector angular range is specified dependent upon the angular range and the central point of the reference respiratory cycle, and whereby on comparison, the vector between the respiratory signal progression and the central point of the reference respiratory cycle and also the vector angular range are used. This embodiment offers as an advantage, in particular, a rapid comparison.

One embodiment provides that the tangential angular range and the vector angular range are specified and that on comparison, the tangent of the respiratory signal progression and the tangential angular range and the vector between the respiratory signal progression and the central point of the reference respiratory cycle and also the vector angular range are used. This embodiment is advantageous, in particular, since the more criteria that are tested, the better, typically, is the data quality of the projection data.

At least one embodiment of the application is directed to a computed tomography system comprising the respiratory detection unit, the planning unit and the control unit. Typically, the computed tomography system comprises the X-ray tube and the X-ray detector. The computed tomography system is preferably configured according to at least one embodiment of the method for carrying out the imaging scan of the patient.

At least one embodiment of the application is directed to a computer program product which is directly loadable into a memory store of the computer unit has program code segments in order to implement at least one embodiment of the method for carrying out the imaging scan of the patient in the computed tomography system when the computer program product is executed in the computer unit. The computer unit can be part of the computed tomography system and/or can comprise the processor and the working memory.

The computer program product can be a computer program or can comprise a computer program. The computer program product has, in particular, the program code segments which form at least one embodiment of the inventive method. By this, at least one embodiment of the inventive method can be carried out in a defined and repeatable manner and monitoring of a passing on of at least one embodiment of the inventive method can be achieved.

The computer program product is preferably configured such that the computer unit can carry out at least one embodiment of the inventive method via the computer program product. The program code segments can be loaded, in particular, into a memory store of the computer unit and are typically carried out via a processor of the computer unit with access to the memory store. If the computer program product, in particular the program code segments, is carried out in the computer unit, typically all the inventive embodiments of the method described can be carried out.

The computer program product is, for example, stored on a physical computer-readable medium and/or digitally as a data packet in a computer network. The computer program product can represent the physical, computer-readable medium and/or the data packet in the computer network. At least one embodiment of the invention can thus also proceed from the physical computer-readable medium and/or the data packet in the computer network.

The physical, computer-readable medium is typically connectable directly to the computer unit, for example, in that the physical computer-readable medium is placed in a DVD drive or inserted into a USB port, whereby the computer unit can access the physical computer-readable medium, in particular readingly. The data packet can preferably be called from the computer network. The computer network can comprise the computer unit or can be indirectly connected via a Wide Area Network (WAN) or a (Wireless) Local Area Network connection (WLAN or LAN) to the computer unit. For example, the computer program product can be stored digitally on a Cloud server at a storage location of the computer network, and transferred via the WAN via the Internet and/or via the WLAN or LAN to the computer unit, in particular by way of the calling of a download link which points to the storage location of the computer program product.

Features, advantages or alternative embodiments mentioned in the description of the device are also transferable similarly to the method and vice versa. In other words, claims for the method can be developed with features of the device and vice versa. In particular, embodiments of the inventive device can be used in the method.

FIG. 1 shows a flow diagram of a method for carrying out an imaging scan of a patient P in a computed tomography system 10 in a first example embodiment.

Method step S100 denotes an acquisition of a respiratory cycle of the patient P via a respiratory detection unit 11 of the computed tomography system 10.

Method step S101 denotes a provision of the respiratory cycle of the patient P in a planning unit 12 of the computed tomography system 10 as a reference respiratory cycle R.

Method step S102 denotes a selection of at least one respiratory phase AP, AP1, AP2 of the reference respiratory cycle R via the planning unit 12.

Method step S103 denotes a transference of the at least one selected respiratory phase AP, AP1, AP2 together with the reference respiratory cycle R into a control unit 13 of the computed tomography system 10.

Method step S104 denotes an execution of the imaging scan in the computed tomography system 10.

Method step S104A denotes that a respiration of the patient P is acquired via the respiratory detection unit 11 and is transferred as a respiratory signal progression ASV into the control unit 13.

Method step S104B denotes that the respiratory signal progression ASV is compared in the control unit 13 with the at least one selected respiratory phase AP, AP1, AP2 of the reference respiratory cycle R, by which a binary comparison result is generated.

Method step S104C denotes that, dependent upon the binary comparison result, a data acquisition is triggered in the computed tomography system 10, by which, projection data is acquired in the at least one respiratory phase AP, AP1, AP2.

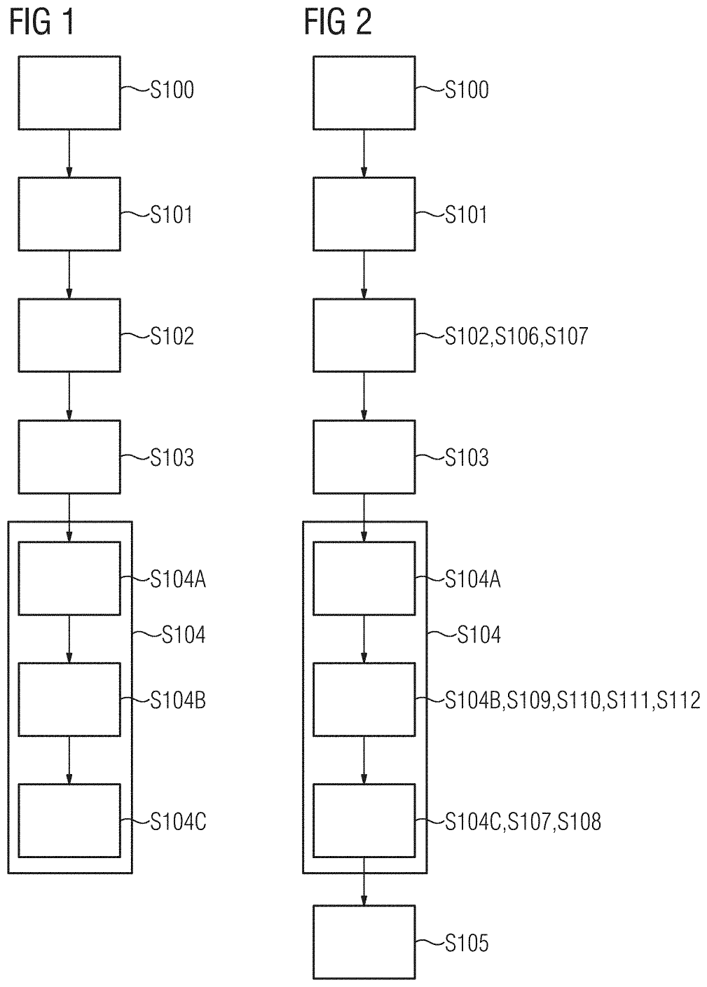

FIG. 2 shows a flow diagram of the method for carrying out the imaging scan of the patient P in the computed tomography system 10 in a second example embodiment.

Method step S105 denotes that after carrying out the imaging scan, a medical image is reconstructed and provided in the at least one respiratory phase AP, AP1, AP2, making use of the projection data.

Method step S106 denotes that the at least one selected respiratory phase AP, AP1, AP2 is shorter than a cycle period of the reference respiratory cycle R.

Method step S107 denotes that the at least one respiratory phase AP, AP1, AP2 comprises a first respiratory phase AP1 and a second respiratory phase AP2 which cover different respiratory phases of the reference respiratory cycle R, and that the data acquisition is triggered according to the first respiratory phase AP1 and according to the second respiratory phase AP2.

Method step S108 denotes that the imaging scan is carried out in a scan region with a first z-position and a second z-position and that the projection data is acquired according to the at least one respiratory phase AP, AP1, AP2 at the first z-position and at the second z-position.

The second example embodiment, in particular method step S107 and method step S108, advantageously show the acquisition of the projection data such that the respiration-correlated volumetric image data set can be reconstructed and provided.

Method step S109 denotes that for the comparison of the reference respiratory cycle R, parameterization takes place cyclically dependent upon a rate of change of the reference respiratory cycle R and an amplitude of the reference respiratory cycle R and that the respiratory signal progression ASV is parameterized cyclically dependent upon a rate of change of the respiratory signal progression ASV and an amplitude of the respiratory signal progression ASV.

Method step S110 denotes that the comparison of the respiratory signal progression ASV with the at least one selected respiratory phase AP, AP1, AP2 of the reference respiratory cycle R comprises a calculation of a tangent TASM of the respiratory signal progression ASV.

Method step S111 denotes that the comparison of the respiratory signal progression ASV with the at least one selected respiratory phase AP, AP1, AP2 of the reference respiratory cycle R comprises a specification of an angular range W1, W2 according to the at least one selected respiratory phase AP, AP1, AP2 of the reference respiratory cycle R and that the angular range W1, W2 is specified dependent upon the rate of change of the reference respiratory cycle R and the amplitude of the reference respiratory cycle R.

Method step S112 denotes that a tangential angular range WTASM is specified dependent upon the angular range W1, W2 about a reference tangent of the reference respiratory cycle R and that on comparison, the tangent TASM of the respiratory signal progression ASV and the tangential angular range WTASM are used.

Alternatively or in addition to method step S110, according to a further method step S113 not shown in FIG. 2, the comparison of the respiratory signal progression ASV with the at least one selected respiratory phase AP, AP1, AP2 of the reference respiratory cycle R can comprise a calculation of a vector VASM between the respiratory signal progression ASV and a central point M of the reference respiratory cycle R, whereby a vector angular range WVASM is specified dependent upon the angular range W1, W2 and the central point M of the reference respiratory cycle R and whereby on comparison, the vector VASM between the respiratory signal progression ASV and the central point M of the reference respiratory cycle R and the vector angular range WVASM are used.

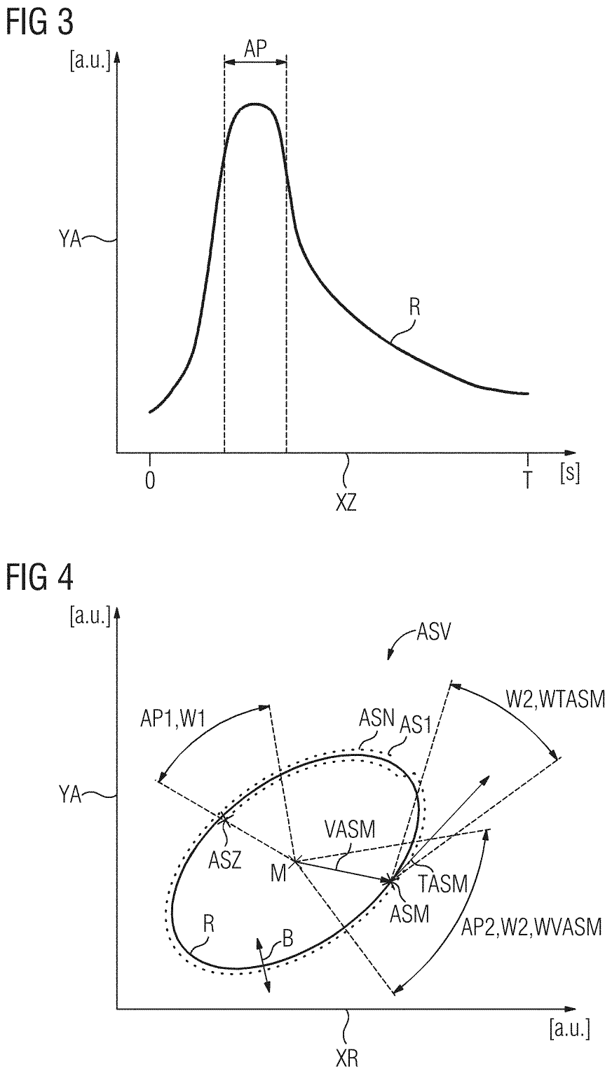

FIG. 3 shows a schematic representation of the reference respiratory cycle R and the at least one selected respiratory phase AP in a third example embodiment. The reference respiratory cycle R can be displayed to the user in this way, for example, on a display unit of the planning unit 12. The reference respiratory cycle R is depicted by way of a y-axis YA with an amplitude of the reference respiratory cycle R and by way of an x-axis XZ with a time progression of the reference respiratory cycle R. The time point T denotes a cycle period of the reference respiratory cycle and corresponds, with a phase angle-dependent parameterization, to 2π. The user can, for example, select the at least one respiratory phase AP via input device(s) of the planning unit 12.

FIG. 4 shows a schematic representation of the reference respiratory cycle R and the at least one selected respiratory phase AP1, AP2 in a fourth example embodiment. FIG. 4 illustrates schematically and preferably at least partially, the subject matter of the method steps S106, S107 and S109 to S113.

The reference respiratory cycle R can be displayed to the user in this way, for example, on a display unit of the planning unit 12. The reference respiratory cycle R is depicted by way of the y-axis YA with the amplitude of the reference respiratory cycle R and by way of an x-axis XR with a rate of change of the reference respiratory cycle R. In this case, the reference respiratory cycle R has the cycle period which amounts to 2π. The reference respiratory cycle R can therefore be depicted as a closed circular function with the continuous line. In addition to the reference respiratory cycle R, the respiratory signal progression ASV is shown cyclically parameterized in FIG. 4. The respiratory signal progression ASV is shown with the dotted line.

Preferably, a divergence between the reference respiratory cycle R and the respiratory signal progression ASV is small. In this example embodiment, the respiratory signal ASV is regular relative to the reference respiratory cycle R and is therefore illustrated without fluctuations in the respiratory signal progression ASV and the divergence is small. If in another example embodiment, a further respiration is irregular, a divergence between a respiratory signal progression of the further respiration and a further reference respiratory cycle typically fluctuates.

The respiratory signal progression ASV comprises a plurality of respiratory signals AS1, ASN, ASM, ASZ, during the imaging scan, which can periodically pass through different respiratory phases of the reference respiratory cycle R. The respiratory signal progression ASV begins with the respiratory signal AS1 and ends with the respiratory signal ASZ. The imaging scan can begin, for example, with the respiratory signal AS1 and end with the respiratory signal ASZ. A sampling rate of the respiratory detection unit 11 typically influences the spacing between the respective respiratory signals.

FIG. 4 shows schematically that the at least one selected respiratory phase AP1, AP2 is shorter than the cycle period of the reference respiratory cycle R, since an angular cover is smaller than the cycle period, that is, less than 2π and that the at least one respiratory phase AP1, AP2 comprises a first respiratory phase AP1 and a second respiratory phase AP2, which cover different respiratory phases of the reference respiratory cycle R.

Shown schematically by way of example in FIG. 4 is that the comparison of the respiratory signal progression ASV with the at least one selected respiratory phase AP1, AP2 of the reference respiratory cycle R comprises a calculation of a tangent TASM of the respiratory signal progression ASV.

Further shown schematically in FIG. 4 is that the comparison of the respiratory signal progression ASV with the at least one selected respiratory phase AP1, AP2 of the reference respiratory cycle R comprises a calculation of a vector VASM between the respiratory signal progression ASV and a central point M of the reference respiratory cycle R.

FIG. 4 shows, also schematically, that the comparison of the respiratory signal progression ASV with the at least one selected respiratory phase AP1, AP2 of the reference respiratory cycle R comprises a specification of an angular range W1, W2 according to the at least one selected respiratory phase AP1, AP2 of the reference respiratory cycle R and that the angular range W1, W2 is specified dependent upon the rate of change of the reference respiratory cycle R and the amplitude of the reference respiratory cycle R. In particular, the angular range W1, W2 can then be defined, dependent upon the rate of change of the reference respiratory cycle R and the amplitude of the reference respiratory cycle R if the reference respiratory cycle R and the respiratory signal progression ASV are cyclically parameterized, by which, in particular, on the same basis as the reference respiratory cycle R, the angular range W1, W2 and the respiratory signal progression ASV are represented. In contrast to FIG. 4, for example, in FIG. 3, the reference respiratory cycle R is not cyclically parameterized.

A tangential angular range WTASM is specified dependent upon the angular range W1, W2 about a reference tangent of the reference respiratory cycle R. The reference tangent of the reference respiratory cycle R is not shown in FIG. 4 for reasons of clarity and typically corresponds to a central line of the tangential angular range WTASM. In the comparison, the tangent TASM of the respiratory signal progression ASV and the tangential angular range WTASM are used.

A vector angular range WTASM is specified dependent upon the angular range W1, W2 and a central point M of the reference respiratory cycle R. In the comparison, the vector VASM between the respiratory signal progression ASV and the central point M of the reference respiratory cycle R, as well as the vector angular range WVASM are used.

In principle, the reference tangents, the tangential angular range WTASM and/or the tangent TASM as well as the vector VASM and the vector angular range WVASM are registered to one another and/or are displaced to the same start point for the comparison.

In this example embodiment, the tangent TASM and the vector VASM are represented relative to the respiratory signal ASM. Dependent upon the respiratory signal ASM, the binary comparison result is True and the data acquisition is triggered. In contrast thereto, the binary comparison result in the case of the respiratory signal ASN is False and no projection data is acquired.

The vector VASM enables, in particular, a determination of a temporal divergence. The tangent TASM enables, in particular, a determination of a divergence in the regularity. In principle, it is conceivable that the temporal divergence and/or the divergence in the regularity are determined by way of the vector VASM and/or the tangent TASM.

The generation of the binary comparison result comprises, in particular, a checking of whether the respiratory signal progression ASV relative to the at least one selected respiratory phase AP1, AP2 fulfills the criteria or not. The criteria comprise, in particular, a tolerance range for a divergence between the respiratory signal progression ASV and the at least one selected respiratory phase AP1, AP2.

The tolerance range can correspond, in principle, to the angular range W1, W2. In this case, the divergence between the respiratory signal progression ASV and the at least one selected respiratory phase AP1, AP2, for example, between the respiratory signal ASM selected, by way of example, in FIG. 4 and a central line of the at least one selected respiratory phase AP1, AP2 is determined. In the comparison, it can be determined, in principle, whether the respiratory signal ASM falls within the angular range W1, W2 of the at least one selected respiratory phase AP1, AP2 or not. Alternatively or additionally, it is conceivable that by way of a start and/or an end of the at least one selected respiratory phase AP1, AP2, it is checked whether the respiratory signal progression ASV relative to the at least one selected respiratory phase AP1, AP2 fulfills the criteria or not. If the respiratory signal progression ASV relative to the at least one selected respiratory phase AP1, AP2 fulfills the criteria, the binary comparison result typically has True and the data acquisition is triggered. In accordance with the respiratory signal progression ASV, projection data is acquired in the first respiratory phase AP1 twice and, in the second respiratory phase AP2, once.

The tolerance range can further comprise a band B pulled radially round the reference respiratory cycle R with a thickness selected, in particular, dependent upon the regularity of the respiration of the patient P, in which the respiratory signal progression ASV is typically found if the binary comparison result has True. For reasons of clarity, the band B is indicated only at one site of the reference respiratory cycle R.

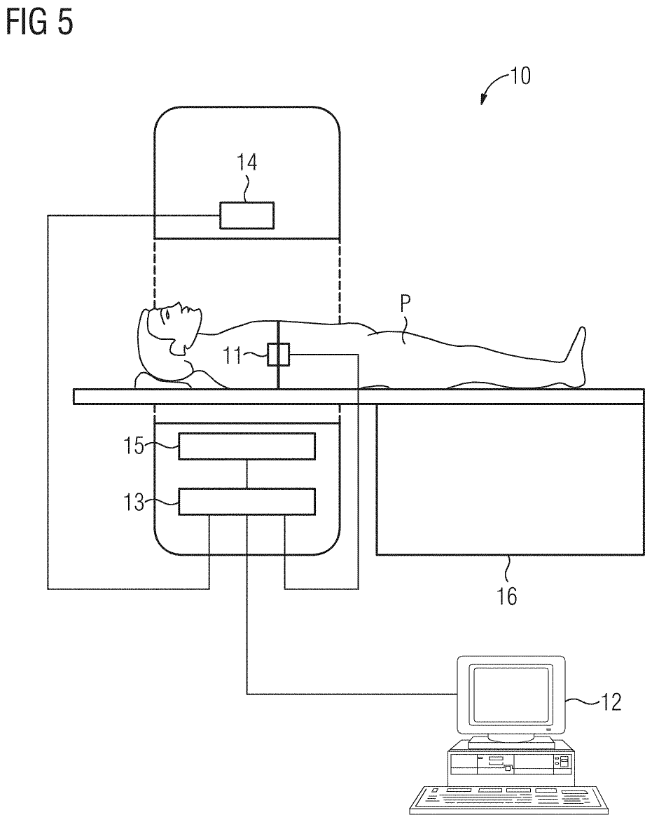

FIG. 5 shows a computed tomography system 10 schematically in cross-section. The computed tomography system 10 comprises the respiratory detection unit 11, the planning unit 12 and the control unit 13. In addition, the computed tomography system 10 comprises an X-ray tube 14 and an X-ray detector 15. For the data acquisition, the X-ray tube 14 is firstly configured for emitting X-ray radiation which, after penetrating a patient P, is detected by the X-ray detector 15 as projection data. The patient P is positioned on a patient support 16.

In this example embodiment, the respiratory detection unit 11 is configured as a respiratory belt. The planning unit 12 has a display unit and an output device(s). The control unit 13 is linked to the respiratory detection unit 11, the planning unit 12, the X-ray tube 14 and the X-ray detector 15, in particular for the acquisition of the respiratory cycle according to method step S100, for the provision of the respiratory cycle according to method step S101, for the selection of the at least one respiratory phase S102, for the transfer of the at least one selected respiratory phase according to method step S103 and for carrying out the imaging scan according to method step S104, S104A, S104B, S104C. A computer unit can be configured as the control unit 13. Although the invention has been illustrated and described in detail with the preferred example embodiments, the invention is not restricted by the examples given and other variations can be derived therefrom by a person skilled in the art without departing from the protective scope of the invention.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for carrying out an imaging scan of a patient in a computed tomography system, comprising:
acquiring a respiratory cycle of the patient via a respiratory detection unit of the computed tomography system,
providing the respiratory cycle of the patient, in a planning unit of the computed tomography system, as a reference respiratory cycle;
selecting, via the planning unit, at least one respiratory phase of the reference respiratory cycle, the at least one respiratory phase selected being relatively shorter than a cycle period of the reference respiratory cycle;
transferring the at least one respiratory phase selected, together with the reference respiratory cycle, into a control unit of the computed tomography system; and
carrying out the imaging scan in the computed tomography system,
a respiration of the patient being acquired via the respiratory detection unit and being transferred as a respiratory signal progression into the control unit,
the respiratory signal progression being compared in the control unit with the at least one respiratory phase selected, of the reference respiratory cycle, by which a binary comparison result is generated, and
wherein, dependent upon a result of the binary comparison, a data acquisition is triggered in the computed tomography system by which projection data is acquired in the at least one respiratory phase.

2. The method of claim 1, wherein, after the imaging scan has been carried out, a medical image is reconstructed and provided in the at least one respiratory phase, making use of the projection data.

3. The method of claim 1, wherein the at least one respiratory phase includes a first respiratory phase and a second respiratory phase, respectively covering different respiratory phases of the reference respiratory cycle, and the data acquisition being triggered according to the first respiratory phase and according to the second respiratory phase.

4. The method of claim 1, wherein the imaging scan is carried out in a scan region with a first z-position and a second z-position and wherein the projection data is acquired according to the at least one respiratory phase at the first z-position and at the second z-position.

5. The method of claim 2, wherein the imaging scan is carried out in a scan region with a first z-position and a second z-position and wherein the projection data is acquired according to the at least one respiratory phase at the first z-position and at the second z-position.

6. The method of claim 1, wherein for comparing of the reference respiratory cycle, parameterization takes place cyclically dependent upon a rate of change of the reference respiratory cycle and an amplitude of the reference respiratory cycle and wherein the respiratory signal progression is parameterized cyclically dependent upon a rate of change of the respiratory signal progression and an amplitude of the respiratory signal progression.

7. The method of claim 1, wherein comparing, of the respiratory signal progression with the at least one respiratory phase of the reference respiratory cycle selected, includes a calculation of a tangent of the respiratory signal progression.

8. The method of claim 1, wherein comparing, of the respiratory signal progression with the at least one respiratory phase of the reference respiratory cycle selected, includes a calculation of a vector between the respiratory signal progression and a central point of the reference respiratory cycle.

9. The method of claim 1, wherein comparing of the respiratory signal progression, with the at least one respiratory phase of the reference respiratory cycle selected, includes a specification of an angular range according to the at least one respiratory phase of the reference respiratory cycle selected, the angular range being specified dependent upon a rate of change of the reference respiratory cycle and an amplitude of the reference respiratory cycle.

10. The method of claim 7, wherein a tangential angular range is specified dependent upon the angular range about a reference tangent of the reference respiratory cycle and wherein, on comparison, the tangent of the respiratory signal progression and the tangential angular range are used.

11. The method of claim 8, wherein a vector angular range is specified dependent upon the angular range and the central point of the reference respiratory cycle, and wherein on comparison, the vector between the respiratory signal progression and the central point of the reference respiratory cycle and also the vector angular range are used.

12. The method of claim 9, wherein a vector angular range is specified dependent upon the angular range and the central point of the reference respiratory cycle, and wherein on comparison, the vector between the respiratory signal progression and a central point of the reference respiratory cycle and also the vector angular range are used.

13. A computed tomography system, comprising:
a respiratory detection unit to acquire a respiratory cycle of a patient;
a planning unit to provide a respiratory cycle of the patient, as a reference respiratory cycle and to select at least one respiratory phase of the reference respiratory cycle, the at least one respiratory phase selected being relatively shorter than a cycle period of the reference respiratory cycle; and
a control unit to receive the at least one respiratory phase selected transferred from the planning unit, together with the reference respiratory cycle and to control carrying out an imaging scan in the computed tomography system, wherein
a respiration of the patient is acquired via the respiratory detection unit and is transferred as a respiratory signal progression into the control unit,
the respiratory signal progression is compared in the control unit with the at least one respiratory phase selected, of the reference respiratory cycle, by which a binary comparison result is generated, and
dependent upon a result of the binary comparison, a data acquisition is triggered in the computed tomography system by which projection data is acquired in the at least one respiratory phase.

14. A non-transitory computer program product, storing a computer program which is directly loadable into a memory store of a computer unit, the computer program including program code segments to carry out the method of claim 1 when the computer program is executed in the computer unit.

15. The method of claim 2, wherein the at least one respiratory phase includes a first respiratory phase and a second respiratory phase, respectively covering different respiratory phases of the reference respiratory cycle, and the data acquisition being triggered according to the first respiratory phase and according to the second respiratory phase.

16. The method of claim 3, wherein the imaging scan is carried out in a scan region with a first z-position and a second z-position and wherein the projection data is acquired according to the at least one respiratory phase at the first z-position and at the second z-position.

17. The method of claim 6, wherein comparing of the respiratory signal progression, with the at least one respiratory phase of the reference respiratory cycle selected, includes a specification of an angular range according to the at least one respiratory phase of the reference respiratory cycle selected, the angular range being specified dependent upon the rate of change of the reference respiratory cycle and the amplitude of the reference respiratory cycle.

18. The method of claim 8, wherein a tangential angular range is specified dependent upon the angular range about a reference tangent of the reference respiratory cycle and wherein, on comparison, the tangent of the respiratory signal progression and the tangential angular range are used.

19. The method of claim 9, wherein a tangential angular range is specified dependent upon the angular range about a reference tangent of the reference respiratory cycle and wherein, on comparison, the tangent of the respiratory signal progression and the tangential angular range are used.

20. A non-transitory computer readable medium, storing a computer program which is directly loadable into a memory store of a computer unit, the computer program including program code segments to carry out the method of claim 1 when the computer program is executed in the computer unit.
